(19) United States
(12) Patent Application Publication
(10) Pub. No.: US 2025/0144165 A1
(43) Pub. Date: May 8, 2025

(54) COMPOSITION FOR PREVENTING OR TREATING RESPIRATORY DISEASES COMPRISING PERSICARIA PUBESCENS EXTRACT AS ACTIVE INGREDIENT

(71) Applicant: JEJU NATIONAL UNIVERSITY INDUSTRY-ACADEMIC COOPERATION FOUNDATION, Jeju-si, Jeju-do (KR)

(72) Inventor: Yong Suk CHUNG, Jeju-si (KR)

(73) Assignee: JEJU NATIONAL UNIVERSITY INDUSTRY-ACADEMIC COOPERATION FOUNDATION, Jeju-si, Jeju-do (KR)

(21) Appl. No.: 18/835,893

(22) PCT Filed: Dec. 7, 2022
(86) PCT No.: PCT/KR2022/019773

§ 371 Date: Aug. 5, 2024

(30) Foreign Application Priority Data
Feb. 4, 2022 (KR) 10-2022-0014624

(51) Int. Cl. A61K 36/70; A61P 11/00

(52) U.S. Cl. CPC A61K 36/70; A61P 11/00

(57) ABSTRACT

The objective of the present invention is to provide a composition for preventing, improving or treating respiratory diseases, comprising a Persicaria pubescens extract as an active ingredient, and the present invention was completed by confirming the effect of the Persicaria pubescens extract on activating the cell viability of human bronchial epithelial cells (BEAS-2B cells).

### TECHNICAL FIELD

The present disclosure relates to a composition for preventing, improving or treating respiratory diseases comprising a Persicaria pubescens extract as an active ingredient.

### BACKGROUND ART

The present disclosure relates to a composition for preventing, improving or treating respiratory diseases comprising a Persicaria pubescens extract as an active ingredient.

Chronic obstructive pulmonary disease (COPD), one of representative respiratory diseases along with asthma, differs from asthma in that irreversible airway obstruction is accompanied, and currently ranks fourth in the world in mortality rate and is only a major disease among the top 10 diseases whose incidence rate is increasing. The COPD is a disease caused by pathological changes in the bronchioles and lung parenchyma due to inflammation of the respiratory tract and lung parenchyma, and is characterized by obstructive bronchitis and emphysema (destruction of lung parenchyma). Types of chronic obstructive pulmonary disease include chronic obstructive bronchitis, chronic bronchiolitis, and emphysema.

Up until now, in the case of COPD including asthma, therapeutic agents having anti-inflammatory or bronchiectatic effects have been used to treat inflammatory diseases. Many of these existing therapeutic agents require caution when used due to many side effects. Representative therapeutic agents include glucocorticoids, leukotriene modifiers, theophylline, etc. Since glucocorticoids are powerful in terms of effectiveness, but do not act selectively, but rather suppress all immune responses and anti-inflammatory responses, in some cases, there is a problem that even necessary immune responses are suppressed, and inhaled treatment is performed due to the problem of drug side effects. Leukotriene modifiers have few side effects, but are limited in effectiveness, and thus may not control asthma when used alone, and has a problem of being mostly used as an adjuvant. Theophylline is not excellent even in effectiveness and has a problem that there is a concern about side effects. Corticosteroid agents show excellent therapeutic effects, but when used in long-term, it is known to cause adrenal suppression, decreased bone density, growth disorders, complications in the eyes and skin, increased collagen synthesis, etc. in proportion to a dose and a duration of use. Long-acting beta-2 agonists such as salmeterol and formoterol have an effect of preventing seizures, but in some cases, have been warned to cause death of patients. Due to these various side effects, conventional therapeutic agents for inflammatory diseases require careful consideration in used thereof, and there is an urgent need for the development of therapeutic agents with an excellent effect and fewer side effects.

Meanwhile, Persicaria pubescens is an annual plant that likes moist places, and has stems that grow straight or obliquely to reach a height of 40 to 80 cm, and has a few hairs in the whole body. Broad lanceolate leaves are arranged alternately on the reddish stem, and the leaf surfaces have black patterns that resemble the number eight ( [symbol] ) in Chinese characters. The ends of the leaves are pointed and the edges thereof are smooth. Other Persicaria plants have a spicy taste when chewing the leaves, but this plant is not spicy, which is called Persicaria pubescens.

The present inventors have conducted continuous research to develop a natural substance capable of preventing lung diseases by enhancing a lung function and protecting the lungs from fine dust and various environmental pollutants, and confirmed that an alcohol extract of Persicaria pubescens had a cell activity effect on human bronchial epithelial cells (BEAS-2B cells), and then completed the present disclosure.

### DISCLOSURE

### Technical Problem

An object of the present disclosure is to provide a pharmaceutical composition for preventing, improving or treating respiratory diseases comprising a Persicaria pubescens extract as an active ingredient.

Another object of the present disclosure is to provide a food composition for preventing or improving respiratory diseases comprising a Persicaria pubescens extract as an active ingredient.

Yet another object of the present disclosure is to provide a cosmetic composition for preventing or improving respiratory diseases comprising a Persicaria pubescens extract as an active ingredient.

Finally, still another object of the present disclosure is to provide a method for treating respiratory diseases including administering the pharmaceutical composition to a subject.

### Technical Solution

An aspect of the present disclosure provides a pharmaceutical composition for preventing or treating respiratory diseases comprising a Persicaria pubescens extract as an active ingredient.

In an example of the present disclosure, the composition of the present disclosure may be extracted with at least one extraction solvent selected from the group consisting of water, anhydrous or hydrous alcohols having 1 to 4 carbon atoms, ethyl acetate, acetone, glycerin, ethylene glycol, propylene glycol, and butylene glycol, preferably alcohol, and more preferably 50% alcohol, but is not limited thereto.

In an example of the present disclosure, the composition of the present disclosure may be any one selected from the group consisting of flowers, leaves, stems, fruits, roots, and mixtures thereof of Persicaria pubescens, but is not limited thereto.

In an example of the present disclosure, the composition of the present disclosure may be a composition for preventing or treating respiratory disease which is any one selected from the group consisting of asthma, allergic rhinitis, bronchitis, and chronic obstructive pulmonary disease, but is not limited thereto.

In an example of the present disclosure, the composition of the present disclosure may increase cell viability of human bronchial epithelial cells (BEAS-2B cells), but is not limited thereto.

In an example of the present disclosure, the composition of the present disclosure may inhibit cleaved-PARP protein expression, but is not limited thereto.

In addition, another aspect of the present disclosure provides a cosmetic composition for preventing or improving respiratory diseases, comprising a Persicaria pubescens extract as an effective ingredient.

In addition, yet another aspect of the present disclosure provides a food composition for preventing or improving respiratory diseases, comprising a Persicaria pubescens extract as an effective ingredient.

Finally, still another aspect of the present disclosure provides a method for treating respiratory diseases comprising administering the pharmaceutical composition to a subject.

### Advantageous Effects

The present disclosure relates to a composition for preventing, improving or treating respiratory diseases comprising a Persicaria pubescens extract as an active ingredient. Specifically, the Persicaria pubescens extract has an excellent effect on activating the cell viability of human bronchial epithelial cells (BEAS-2B cells) and thus can be effectively used as a pharmaceutical composition for preventing or treating respiratory diseases, a cosmetic composition for preventing or improving respiratory diseases, or a food composition for preventing or improving respiratory diseases.

### DESCRIPTION OF DRAWINGS

FIG. 1 is a diagram illustrating Persicaria pubescens of the present disclosure.

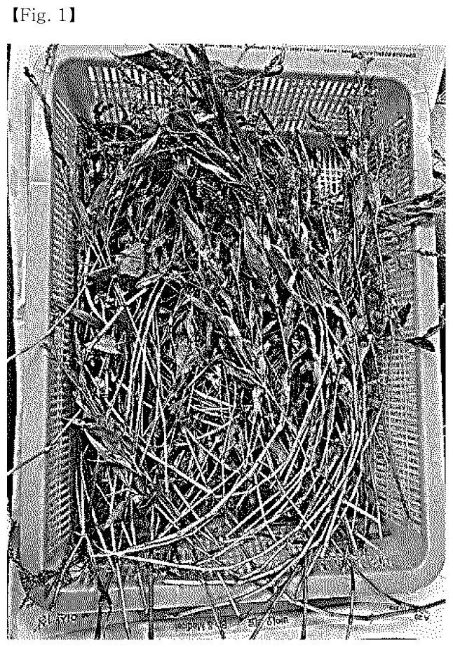

FIG. 2 is a diagram illustrating an effect of a Persicaria pubescens extract (PPE) of the present disclosure on activating the cell viability of human bronchial epithelial cells (BEAS-2B cells).

FIG. 3 is a diagram illustrating a result of inhibiting the expression of cleaved-PARP, an apoptotic factor of human bronchial epithelial cells (BEAS-2B cells) by a Persicaria pubescens extract (PPE) of the present disclosure.

### BEST MODE OF THE INVENTION

Hereinafter, exemplary embodiments of the present disclosure will be described in detail with reference to the accompanying drawings. However, the following exemplary embodiments are presented as examples for the present disclosure, and when it is determined that a detailed description of well-known technologies or configurations known to those skilled in the art may unnecessarily obscure the gist of the present disclosure, the detailed description thereof may be omitted, and the present disclosure is not limited thereto. Various modifications and applications of the present disclosure are possible within the description of claims to be described below and the equivalent scope interpreted therefrom.

Further, terminologies used in the present specification are terminologies used to properly express preferred exemplary embodiments of the present disclosure, which may vary according to users, an operator's intention, or customs in the art to which the present disclosure pertains. Accordingly, definitions of the terminologies need to be described based on contents throughout this specification. Throughout the specification, unless explicitly described to the contrary, when a certain part “comprises” an element, will be understood to imply the inclusion of stated elements but not the exclusion of any other elements.

Throughout this specification, ‘%’ used to indicate the concentration of a specific material is solid/solid (w/w) %, solid/liquid (w/v) %, and liquid/liquid (v/v) %, unless otherwise stated.

In an aspect, an object of the present disclosure is to provide a pharmaceutical composition for preventing or treating respiratory diseases comprising a Persicaria pubescens extract as an active ingredient.

The part of Persicaria pubescens used in the Persicaria pubescens extract of the present disclosure may be any one selected from the group consisting of flowers, leaves, stems, fruits, roots, and mixtures thereof. Preferably, in the present disclosure, it was confirmed that obtaining an extract from the leaves of Persicaria pubescens is more effective in preventing or treating respiratory diseases than using other parts, and thus an extract from the leaves of Persicaria pubescens was used.

The extract according to the present disclosure may be obtained and used by extraction and separation from the nature using extraction and separation methods known in the art, and the “extract” as defined in the present disclosure is extracted from Persicaria pubescens using a suitable solvent, and includes all, for example, a crude extract, a polar solvent-soluble extract, or a non-polar solvent-soluble extract. As the suitable solvent for extracting the extract from the Persicaria pubescens, any cytologically or pharmaceutically acceptable organic solvent may be used, and water or an organic solvent may be used, but is not limited thereto. For example, various solvents such as purified water, alcohols having carbon atoms 1 to 4 including methanol, ethanol, propanol, isopropanol, butanol, etc., acetone, ether, benzene, chloroform, ethyl acetate, methylene chloride, hexane, cyclohexane, and the like may be used alone or in combination. As the extraction method, any one of methods such as hot water extraction, chilling extraction, reflux cooling extraction, solvent extraction, steam distillation, ultrasonic extraction, elution, and compression may be selected and used. In addition, a desired extract may also be additionally subjected to a conventional fractionation process or purified using a conventional purification method.

There is no limitation to a preparation method of the extract of the present disclosure, and any known preparation method may be used. For example, the extract included in the composition of the present disclosure may be prepared with a primary extract extracted by the hot water extraction or solvent extraction method in a powder state by additional processes such as distilling under reduced pressure and freeze-drying or spray-drying. In addition, the primary extract may be extracted again using an appropriate solvent to prepare a secondary extract, and fractions may also be obtained by further purifying the primary extract using various chromatography, such as silica gel column chromatography, thin layer chromatography, and high performance liquid chromatography. Accordingly, in the present disclosure, the extract is a concept that includes all extracts, and fractioned and purified products that are obtained in each step of extraction, fractionation, or purification, and dilutions, concentrates, or dried products thereof.

The pharmaceutical composition of the present disclosure may further include an adjuvant in addition to the active ingredient. The adjuvant may be used with any adjuvant known in the art without limitation, but further include, for example, a Freund's complete adjuvant or an incomplete adjuvant to increase the effect of the present disclosure.

The pharmaceutical composition according to the present disclosure may be prepared in the form of incorporating the active ingredient into a pharmaceutically acceptable carrier. Here, the pharmaceutically acceptable carrier includes carriers, excipients and diluents commonly used in a pharmaceutical field. The pharmaceutically acceptable carrier that may be used in the pharmaceutical composition of the present disclosure is not limited thereto, but may include lactose, dextrose, sucrose, sorbitol, mannitol, xylitol, erythritol, maltitol, starch, acacia gum, alginate, gelatin, calcium phosphate, calcium silicate, cellulose, methyl cellulose, polyvinylpyrrolidone, water, methyl hydroxybenzoate, propyl hydroxybenzoate, talc, magnesium stearate, and mineral oil.

The pharmaceutical composition of the present disclosure may be formulated and used in the form of oral formulations, such as powders, granules, tablets, capsules, suspensions, emulsions, syrups, aerosols, etc., external preparations, suppositories, or sterile injectable solutions according to each conventional method.

The formulations may be prepared by using diluents or excipients, such as a filler, an extender, a binder, a wetting agent, a disintegrating agent, a surfactant, etc., which are generally used. Solid formulations for oral administration include tablets, pills, powders, granules, capsules, etc., and these solid formulations may be prepared by mixing at least one or more excipients, for example, starch, calcium carbonate, sucrose, lactose, gelatin, etc. with the active ingredients. Further, lubricants such as magnesium stearate and talc may be used in addition to simple excipients. Liquid formulations for oral administration may correspond to suspensions, oral liquids, emulsions, syrups, etc., and may include various excipients, for example, a wetting agent, a sweetener, an aromatic agent, a preserving agent, etc., in addition to the commonly used diluents, such as water and liquid paraffin. Formulations for parenteral administration include sterile aqueous solutions, non-aqueous solvents, suspensions, emulsions, lyophilized agents, and suppositories. As the non-aqueous solution and the suspension, propylene glycol, polyethylene glycol, vegetable oil such as olive oil, injectable ester such as ethyl oleate, etc. may be used. As the base material of the suppository, witepsol, Tween 61, cacao butter, laurinum, glycerogelatin, etc. may be used.

The pharmaceutical composition according to the present disclosure may be administered to a subject through various routes. All methods of administration may be expected, and the pharmaceutical composition may be administered by, for example, oral, intravenous, intramuscular, subcutaneous, and intraperitoneal injection.

The pharmaceutical composition may be formulated into various oral or parenteral administration forms.

Formulations for oral administration include, for example, tablets, pills, hard and soft capsules, solutions, suspensions, emulsifiers, syrups, granules, etc., and these formulations may further include diluents (e.g., lactose, dextrose, sucrose, mannitol, sorbitol, cellulose and/or glycine), lubricants (e.g., silica, talc, stearic acid and its magnesium or calcium salts and/or polyethylene glycol), in addition to the active ingredient. In addition, the tablets may contain a binder such as magnesium aluminum silicate, starch paste, gelatin, tragacanth, methylcellulose, sodium carboxymethylcellulose and/or polyvinylpyrrolidine, and in some cases, may contain disintegrants or effervescent mixtures such as starch, agar, alginic acid or its sodium salt and/or an absorbent, a coloring agent, a flavoring agent and a sweetening agent. The formulations may be prepared by conventional mixing, granulating or coating methods.

In addition, a typical formulation for parenteral administration is an injection formulation, and as a solvent for the injection formulation, water, a Ringer's solution, isotonic physiological saline or a suspension may be used. Sterile fixed oils of the injection formulation may be used as a solvent or suspension medium, and any non-irritating fixed oil including mono- and di-glycerides may be used for this purpose.

In addition, the injection formulation may use fatty acids such as oleic acid.

In an aspect, an object of the present disclosure is to provide a cosmetic composition for preventing or improving respiratory diseases comprising a Persicaria pubescens extract as an active ingredient.

The “cosmetic composition for improving” of the present disclosure may be prepared by including a cosmetically effective amount of the extract extracted from the Persicaria pubescens of the present disclosure described above and a cosmetically acceptable carrier.

As used herein, the term “cosmetically effective amount” means an amount sufficient to achieve skin regeneration efficacy through proliferation of epidermal keratinocytes of the composition of the present disclosure as described above.

The appearance of the cosmetic composition contains a cosmetically or dermatologically acceptable medium or base. The appearance of the cosmetic composition may be provided in all formulations suitable for local application, for example, solutions, gels, solids, pasty anhydrous products, emulsions obtained by dispersing an oil phase in a water phase, suspensions, microemulsions, microcapsules, microgranules or ionic (liposomes) and non-ionic vesicular dispersions, or creams, toners, lotions, powders, ointments, sprays or concealer sticks. These compositions may be prepared according to conventional methods in the art. The composition according to the present disclosure may also be used in the form of foam or in the form of an aerosol composition further containing a compressed propellant.

The cosmetic composition according to an example of the present disclosure is not particularly limited in its formulation, and may be formulated in cosmetics, such as softening toner, astringent toner, nourishing toner, nourishing cream, massage cream, essence, eye cream, eye essence, cleansing cream, cleansing foam, cleansing water, pack, powder, body lotion, body cream, body oil, and body essence.

When the formulation of the cosmetic composition of the present disclosure is the paste, cream, or gel, as the carrier ingredient, animal oils, vegetable oils, wax, paraffin, starch, tragacanth, cellulose derivatives, polyethylene glycol, silicone, bentonite, silica, talc, zinc oxide or the like may be used.

When the formulation of the cosmetic composition of the present disclosure is the powder or spray, as the carrier ingredient, lactose, talc, silica, aluminum hydroxide, calcium silicate or polyamide powder may be used, and particularly, in the case of the spray, a propellant such as chlorofluorohydrocarbon, propane/butane or dimethyl ether may be additionally included.

When the formulation of the cosmetic composition of the present disclosure is the solution or emulsion, as the carrier ingredient, a solvent, a solubilizing agent or an emulsifying agent may be used. For example, the carrier ingredient includes water, ethanol, isopropanol, ethyl carbonate, ethyl acetate, benzyl alcohol, benzyl benzoate, propylene glycol, 1,3-butyl glycol oil, glycerol aliphatic ester, polyethylene glycol or fatty acid ester of sorbitan.

When the formulation of the cosmetic composition of the present disclosure is the suspension, as the carrier ingredient, a liquid diluent such as water, ethanol or propylene glycol, a suspension such as ethoxylated isostearyl alcohol, polyoxyethylene sorbitol ester and polyoxyethylene sorbitan ester, microcrystalline cellulose, aluminum metahydroxide, bentonite, agar, tragacanth, or the like may be used.

When the formulation of the cosmetic composition of the present disclosure is the surfactant-containing cleansing, as the carrier ingredient, aliphatic alcohol sulfate, aliphatic alcohol ether sulfate, sulfosuccinic acid monoester, isethionate, imidazolinium derivatives, methyltaurate, sarcosinate, fatty acid amide ether sulfate, alkylamido betaine, aliphatic alcohol, fatty acid glyceride, fatty acid diethanolamide, vegetable oil, lanolin derivatives, ethoxylated glycerol fatty acid ester, or the like may be used.

The cosmetic composition of the present disclosure may be applied to cosmetics, such as skin, lotion, cream, essence, pack, foundation, color cosmetics, sunscreen, two-way cake, face powder, compact, makeup base, skin cover, eye shadow, lipstick, lip gloss, lip fix, and eyebrow pencil, and toner, and cleaning agents, such as shampoo and soap.

The cosmetic composition according to an embodiment of the present disclosure may further include functional additives and ingredients included in general cosmetic compositions in addition to the extract extracted from the Persicaria pubescens. The functional additives may include ingredients selected from the group consisting of water-soluble vitamins, oil-soluble vitamins, polymer peptides, polymer polysaccharides, sphingolipids, and seaweed extract.

In addition to the functional additives, the cosmetic composition of the present disclosure may also be mixed with ingredients included in general cosmetic compositions, if necessary. Other mixed ingredients to be included may include oil and fat ingredients, moisturizers, emollients, surfactants, organic and inorganic pigments, organic powders, ultraviolet absorbers, preservatives, disinfectants, antioxidants, plant extracts, pH adjusters, alcohols, pigments, fragrances, blood circulation accelerators, cooling agents, adiaphoretics, purified water, etc.

In an aspect, an object of the present disclosure is to provide a food composition for preventing or improving respiratory diseases comprising a Persicaria pubescens extract as an active ingredient.

In addition to comprising the extract as the active ingredient, the food composition of the present disclosure may include various flavoring agents or natural carbohydrates as additional ingredients, like conventional food compositions.

Examples of the above-described natural carbohydrates include conventional sugars, including monosaccharides, such as glucose, fructose, etc.; disaccharides, such as maltose, sucrose, etc.; and polysaccharides, such as dextrin, cyclodextrin, etc., and sugar alcohols such as xylitol, sorbitol, erythritol, etc. The above-described flavoring agents may be advantageously used with natural flavoring agents (thaumatin), stevia extracts (e.g., rebaudioside A, glycyrrhizin, etc.), and synthetic flavoring agents (saccharin, aspartame, etc.). The food composition of the present disclosure may be formulated in the same manner as the pharmaceutical composition to be used as a functional food or added to various foods. The foods capable of adding the composition of the present disclosure include, for example, beverages, meat, chocolate, foods, confectionery, pizza, ramen, other noodles, gums, candies, ice creams, alcohol beverages, vitamin complexes, health food supplements, etc.

In addition, the food composition may include various nutrients, vitamins, minerals (electrolytes), flavoring agents such as synthetic and natural flavoring agents, coloring agents and enhancers (cheese, chocolate, etc.), pectic acid and salts thereof, alginic acid and salts thereof, organic acid, a protective colloidal thickener, a pH adjusting agent, a stabilizer, a preservative, glycerin, alcohol, a carbonic acid agent used in a carbonated drink, and the like, in addition to the extract as the active ingredient. In addition, the food composition of the present disclosure may contain pulps for preparing natural fruit juice, fruit juice beverages, and vegetable beverages.

The food composition for improving of the present disclosure may be produced and processed in the form of tablets, capsules, powders, granules, liquids, and pills. In the present disclosure, the ‘health functional food composition’ refers to foods prepared and processed by using raw materials or ingredients with functionality, which are useful for the human body according to the Art on Health Functional Foods No. 6727, and means foods taken for adjusting nutrients for the structures and functions of the human body or obtaining a useful effect on health applications such as physiological actions. The food composition of the present disclosure may include conventional food additives, and the suitability as the food additives is determined by the specifications and standards for the corresponding item in accordance with the general rules of the Food Additive Codex, general test methods, and the like approved by the Food and Drug Administration, unless otherwise specified. The items disclosed in the “Food Additives Codex” may include, for example, chemical composites such as ketones, glycine, calcium citrate, nicotinic acid, cinnamic acid, etc.; natural additives such as persimmon color, licorice extract, crystal cellulose, Kaoliang color, guar gum, etc.; mixed formulations such as sodium L-glutamic acid formulations, noodle additive alkali agents, preservative formulations, tar color formulations, etc. For example, the food composition in the form of tablets may be formed by granulating a mixture obtained by mixing the active ingredient of the present disclosure with an excipient, a binder, a disintegrant, and other additives, and then compression-molding the mixture by adding a slip modifier and the like, or directly compressing the mixture. In addition, the food composition in the form of tablets may contain a flavors enhancer or the like as needed. In the food composition in the form of capsules, hard capsules may be prepared by filling a mixture mixed with the active ingredient of the present disclosure and additives such as excipients into conventional hard capsules, and soft capsules may be prepared by filling a mixture mixed with the active ingredient of the present disclosure and additives such as excipients into capsule bases such as gelatin. The soft capsules may contain a plasticizer such as glycerin or sorbitol, a colorant, a preservative, and the like, if necessary. The food composition in the form of pills may be prepared by molding a mixture obtained by mixing the active ingredient of the present disclosure with an excipient, a binder, a disintegrant, etc. by conventional known methods, and may also be coated with white sugar or other coating agents or surface-coated with materials such as starch and talc, if necessary. The food composition in the form of granules may be prepared by granulizing a mixture mixed with the active ingredient of the present disclosure and an excipient, a binder, a disintegrant, etc. by conventional known methods and may contain a flavoring agent, a flavors enhancer, etc., if necessary.

### MODES OF THE INVENTION

Hereinafter, the present disclosure will be described in more detail with reference to Examples. However, the Examples and Experimental Examples are presented as examples for the present disclosure, and when it is determined that a detailed description of well-known technologies or configurations known to those skilled in the art may unnecessarily obscure the gist of the present disclosure, the detailed description thereof may be omitted, and the present disclosure is not limited thereto. Various modifications and applications of the present disclosure are possible within the description of claims to be described below and the equivalent scope interpreted therefrom.

### EXAMPLE

### Example 1. Preparation of Persicaria pubescens Extract

Persicaria pubescens produced in the Jeju Island was washed and naturally dried for 7 days, and the dried Persicaria pubescens was ground and sieved through a 1.70 mm sieve. Thereafter, 50% alcohol was extracted for 2 hours at a ratio of 1:10 for the dried material and a solvent, and the extracted alcohol extract was centrifuged at 2000 RPM for 5 minutes, and then the supernatant was filtered under reduced pressure and concentrated at 55° C. (Rotary Evaporator). Then, the concentrate was freeze-dried at −80° C. and 5 mTorr for 72 hours to produce a powder form. The powder was dissolved in DMSO at a concentration of 100 mg/mL and used.

### Example 2. Experiment for Confirming Cell Viability and Apoptosis Factor Inhibition Effect of Persicaria pubescens Extract on Human Bronchial Epithelial Cells (BEAS-2B Cells)

### 2-1. Experimental Method

To evaluate an apoptosis inhibition effect of a Persicaria pubescens extract on human bronchial epithelial cells (BEAS-2B cells), the Persicaria pubescens extract was pretreated for 1 hour at concentrations of 25, 50, and 100 μg/mL, respectively, and then treated with 50 μM 1-Nitropyrene for 24 hours to induce apoptosis. Thereafter, a (3-(4,5-dimethylthiazol-2-yl)-2,5-diphenyltetrazolium bromide (MTT) reagent was treated for 2 hours, and the absorbance was measured at 595 nm.

In addition, in order to evaluate the apoptosis inhibition activity of the Persicaria pubescens extract on human bronchial epithelial cells, the expression level of cleaved-PARP as an apoptosis factor was measured.

The human bronchial epithelial cells, BEAS-2B cells, were treated with the Persicaria pubescens extract at concentrations of 50 and 100 μg/mL for 1 hour, and then treated with 50 μM 1-Nitropyrene for 24 hours, and the cells were lysed and harvested. Thereafter, a certain amount of proteins was included through protein quantification, and the expression level of cleaved-PARP was evaluated by western blot, which was a method used to detect specific protein molecules in a protein mixture.

In the western blot for evaluating the expression level, protein molecules were separated by size using gel electrophoresis, and then the proteins were transferred from a gel to a membrane, the membrane was incubated with a primary antibody specifically binding to cleaved-PARP and reacted, and then incubated with a secondary antibody that specifically recognized and bound to the primary antibody. The secondary antibody was easily detected by generating light, and through this step, the expression level of cleaved-PARP in the protein mixture was detected, and the expression level was quantified using an image J software program.

### 2-2. Experiment Results

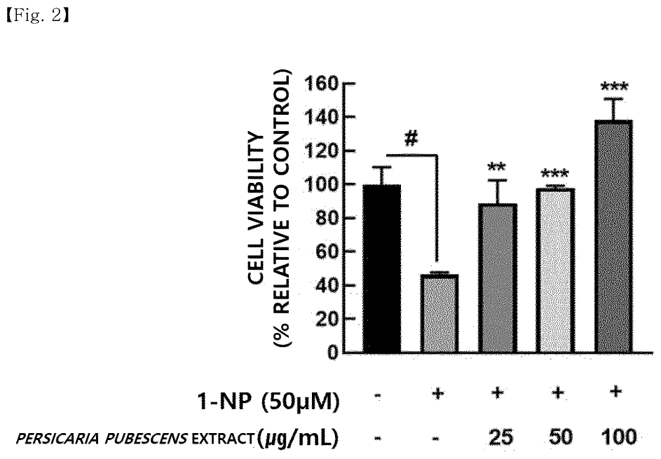

As a result, as illustrated in FIG. 2, it was confirmed that the cell viability of human bronchial epithelial cells (BEAS-2B cells), which was decreased by treatment of a control group (1-Nitropyrene), significantly increased by treatment of a Persicaria pubescens extract.

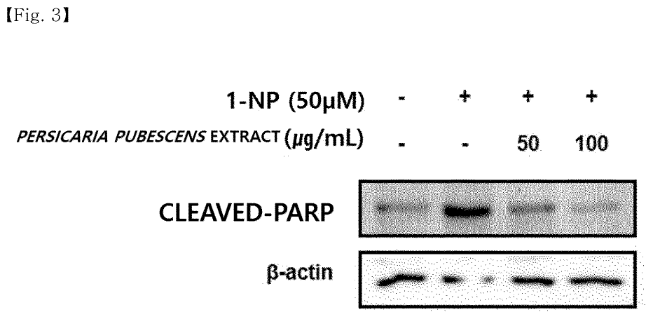

In addition, as illustrated in FIG. 3, it was confirmed that the expression of cleaved-PARP, a factor related to the death of human bronchial epithelial cells (BEAS-2B cells), was reduced by the treatment of the Persicaria pubescens extract.

From these results, it is determined that the Persicaria pubescens extract has an effect of preventing or treating respiratory diseases and may be used as a composition for preventing or treating respiratory diseases.

The present disclosure has been described above with reference to preferred embodiments thereof.

It will be understood to those skilled in the art that the present disclosure may be implemented as modified forms without departing from an essential characteristic of the present disclosure. Therefore, the disclosed exemplary embodiments should be considered in an illustrative viewpoint rather than a restrictive viewpoint. The scope of the present disclosure is illustrated by the appended claims rather than by the foregoing description, and all differences within the scope of equivalents thereof should be construed as being included in the present disclosure.

1. A method for preventing or treating a respiratory disease, comprising administering a pharmaceutical composition comprising a Persicaria pubescens extract as an active ingredient to a subject in need thereof.

2. The method of claim 1, wherein the Persicaria pubescens extract is extracted with at least one extraction solvent selected from the group consisting of water, anhydrous or hydrous alcohols having 1 to 4 carbon atoms, ethyl acetate, acetone, glycerin, ethylene glycol, propylene glycol, and butylene glycol.

3. method of claim 1, wherein the Persicaria pubescens extract is derived from at least one parts selected from the group consisting of flowers, leaves, stems, fruits, roots, and mixtures thereof.

4. The method of claim 1, wherein the respiratory disease is any one selected from the group consisting of asthma, allergic rhinitis, bronchitis, and chronic obstructive pulmonary disease.

5. The method of claim 1, wherein the subject requires increasing cell viability of human bronchial epithelial cells (BEAS-2B cells).

6. The method of claim 1, wherein the subject requires inhibiting expression of cleaved-PARP protein.

7. A method for preventing or improving respiratory diseases, comprising applying a cosmetic composition comprising a Persicaria pubescens extract as an effective ingredient to a subject in need thereof.

8. A method for preventing or improving respiratory diseases, comprising administering a food composition comprising a Persicaria pubescens extract as an effective ingredient to a subject in need thereof.

9. (canceled)
